SEQUENCE LISTING (12 sequences)

SEQ ID NO: 1 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 278 aa, Macaca fascicularis
MVRLPLQCVLWGCLLTAVYPEPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQRALVVIPICLGILFVILLLVLVFIKKVAKKPNDKVPHPKQEPQEINFPDDLPGSNPAAPVQETLHGCQPVTQEDGKESRISVQERQ

SEQ ID NO: 3 - protein, 214 aa, Artificial Sequence
  Synthetic Polypeptide
DIQMTQSPSSLSASVGDRVTISCRASQDISNYLNWYQQKPGKVPKLLIYFTSRLRSGVPSRFSGSGSGTDYTLTISSLQPEDVATYYCQQDRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 443 aa, Artificial Sequence
  Synthetic Polypeptide
EVQLVQSGAEVKKPGASVKVSCKASGYTFTTFPIEWVRQAPGQGLEWMGNFHPYNDDTKYNEKFKGRVTLTADKSTSTAYMELSRLRSEDTAVYYCARRGKLPFDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 5 - protein, 107 aa, Artificial Sequence
  Synthetic Polypeptide
DIQMTQSPSSLSASVGDRVTISCRASQDISNYLNWYQQKPGKVPKLLIYFTSRLRSGVPSRFSGSGSGTDYTLTISSLQPEDVATYYCQQDRKLPWTFGQGTKLEIK

SEQ ID NO: 6 - protein, 117 aa, Artificial Sequence
  Synthetic Polypeptide
EVQLVQSGAEVKKPGASVKVSCKASGYTFTTFPIEWVRQAPGQGLEWMGNFHPYNDDTKYNEKFKGRVTLTADKSTSTAYMELSRLRSEDTAVYYCARRGKLPFDSWGQGTTVTVSS

SEQ ID NO: 7 - protein, 11 aa, Artificial Sequence
  Synthetic Polypeptide
RASQDISNYLN

SEQ ID NO: 8 - protein, 7 aa, Artificial Sequence
  Synthetic Polypeptide
FTSRLRS

SEQ ID NO: 9 - protein, 9 aa, Artificial Sequence
  Synthetic Polypeptide
QQDRKLPWT

SEQ ID NO: 10 - protein, 5 aa, Artificial Sequence
  Synthetic Polypeptide
TFPIE

SEQ ID NO: 11 - protein, 17 aa, Artificial Sequence
  Synthetic Polypeptide
NFHPYNDDTKYNEKFKG

SEQ ID NO: 12 - protein, 8 aa, Artificial Sequence
  Synthetic Polypeptide
RGKLPFDS

(19) United States
(12) Patent Application Publication
(10) Pub. No.: US 2018/0264112 A1
(43) Pub. Date: Sep. 20, 2018

(54) Anti-CD40 Antibody Formulations

(71) Applicant: Biogen MA Inc., Cambridge, MA (US)

(72) Inventors: Mark Krebs, Arlington, MA (US); Adnan Zunic, Cambridge, MA (US); Tia Estey, Swampscott, MA (US); Shantanu Sule, Arlington, MA (US); Margaret McGrath, Somerville, MA (US); Lori Karpes, Quincy, MA (US); Yana Kravets, Salem, MA (US)

(21) Appl. No.: 15/543,653

(22) PCT Filed: Jan. 15, 2016
(86) PCT No.: PCT/US2016/013627

§ 371 Date: Jul. 14, 2017

Related U.S. Application Data
(60) Provisional application No. 62/104,801, filed on Jan. 18, 2015.

(51) Int. Cl. A61K 39/395; C07K 16/28; A61K 47/12; A61K 47/18; A61K 47/26; A61K 9/00; A61K 47/22

(52) U.S. Cl. CPC A61K 39/39591; C07K 16/2878; A61K 47/12; A61K 2039/54; A61K 47/26; A61K 9/0019; A61K 47/22; A61K 47/183

(57) ABSTRACT

Provided herein, in some aspects, are low viscosity, low aggregation propensity, high anti-CD40 antibody concentration formulations.

### RELATED APPLICATIONS

This application claims the benefit under 35 U.S.C. § 119(e) of U.S. provisional application No. 62/104,801, filed Jan. 18, 2015, which is incorporated by reference herein in its entirety.

### FIELD OF THE INVENTION

The present disclosure relates, in some aspects, to the field of therapeutic antibody formulations.

### BACKGROUND OF INVENTION

CD40 is a tumor necrosis factor receptor superfamily member expressed broadly on antigen-presenting cells such as dendritic cells, B cells, and monocytes as well as many non-immune cells and a range of tumors. CD40 is over-expressed by greater than 50% of carcinomas and melanomas, and nearly 100% of hematological B-cell malignancies. Anti-CD40 antibodies can be used to treat such cancers. Therapeutic anti-CD40 antibodies require delivery by injection or infusion via an intravenous route, or by subcutaneous injection. For subcutaneous delivery, in particular, it is important that the antibody formulation have a low viscosity, as increases in viscosity create challenges in injectability. In addition to increased viscosity, at higher concentrations, anti-CD40 antibodies also exhibit characteristic problems including aggregation, precipitation and gelation. Thus, development of high anti-CD40 antibody concentration formulations results in several manufacturing, stability, analytical and delivery challenges.

### SUMMARY OF INVENTION

The present disclosure provides, in some aspects, anti-CD40 antibody formulations having a high antibody concentration, low viscosity, and low aggregation propensity such that they are suitable for subcutaneous injections as well as for other routes of administration. Surprisingly, results provided herein show that both viscosity and protein aggregation were at lowest values within a narrow range of pH values—pH 5.0 to pH 6.5—for a formulation containing an anti-CD40 antibody concentration of 150 mg/ml to 250 mg/ml. Thus, some aspects of the present disclosure provide anti-CD40 antibody formulations that comprise at least 150 mg/ml (or at least 200 mg/ml or at least 250 mg/ml) anti-CD40 antibody formulated at a pH value of 5.5 to 6.0 (e.g., 5.5, 5.6, 5.7, 5.8, 5.9 or 6.0).

In some embodiments, anti-CD40 antibody formulations of the present disclosure comprise one or more of citrate buffer, arginine and sucrose. In some embodiments, anti-CD40 antibody formulations of the present disclosure comprise one or more of histidine buffer, arginine and sucrose.

Thus, some aspects of the present disclosure provide anti-CD40 antibody formulations that comprise at least 150 mg/ml anti-CD40 antibody, 20 mM buffer containing sodium citrate and citric acid, and 150 mM arginine, wherein the formulation has a pH value of 5.5 to 6.0. Some aspects of the present disclosure provide anti-CD40 antibody formulations that comprise at least 150 mg/ml anti-CD40 antibody, 20 mM buffer containing histidine, and 150 mM arginine, wherein the formulation has a pH value of 5.0 to 6.5 (e.g., 5.2-6.2). In some embodiments, the anti-CD40 antibody formulations further comprise 300 mM sucrose.

Some aspects of the present disclosure provide anti-CD40 antibody formulations that comprise 100 mg/ml to 250 mg/ml anti-CD40 antibody, and buffer having a pH value of 5.5 to 6.0. Some aspects of the present disclosure provide anti-CD40 antibody formulations that comprise 100 mg/ml to 250 mg/ml anti-CD40 antibody, and buffer having a pH value of 5.5 to 6.5.

Some aspects of the present disclosure provide anti-CD40 antibody formulations that have a pH value of 5.0±0.5, 5.5±0.5, 6.0±0.5, 6.5±0.5 or 7.0±0.5.

In some embodiments, the buffer is a citrate buffer. In some embodiments, the citrate buffer has a concentration of 10 mM to 30 mM. In some embodiments, the citrate buffer has a concentration of 20 mM.

In some embodiments, the buffer is a histidine buffer. In some embodiments, the histidine buffer has a concentration of 10 mM to 30 mM. In some embodiments, the histidine buffer has a concentration of 20 mM.

In some embodiments, the anti-CD40 antibody formulations further comprise arginine. In some embodiments, arginine is present at a concentration of 140 mM to 160 mM. In some embodiments, arginine is present at a concentration of 150 mM.

In some embodiments, the anti-CD40 antibody formulations further comprise sucrose. In some embodiments, sucrose is present at a concentration of 200 mM to 400 mM. In some embodiments, sucrose is present at a concentration of 300 mM.

Some aspects of the present disclosure provide anti-CD40 antibody formulations that comprise anti-CD40 antibody, 10 mM to 30 mM citrate, and 140 mM to 160 mM arginine, wherein the formulations in solution have a pH value of 5.5 to 6.0. Some aspects of the present disclosure provide anti-CD40 antibody formulations that comprise anti-CD40 antibody, 10 mM to 30 mM histidine, and 140 mM to 160 mM arginine, wherein the formulations in solution have a pH value of 5.0 to 6.5 (e.g., 5.2-6.2).

In some embodiments, the formulations have an anti-CD40 antibody concentration of 100 mg/ml to 300 mg/ml.

In some embodiments, the formulations have an anti-CD40 antibody concentration of at least 150 mg/ml. In some embodiments, the formulations have an anti-CD40 antibody concentration of at least 200 mg/ml. In some embodiments, the formulations have an anti-CD40 antibody concentration of at least 250 mg/ml. In some embodiments, the formulations have an anti-CD40 antibody concentration of 250 mg/ml.

In some embodiments, the anti-CD40 antibody is a monoclonal antibody.

In some embodiments, the formulations have a citrate concentration of 20 mM or a histidine concentration of 20 mM.

In some embodiments, the formulations have an arginine concentration of 150 mM.

In some embodiments, the formulations have a pH value of 5.6-5.8. In some embodiments, the formulations have a pH value of 5.7. In some embodiments, the formulations have a pH value of 6.6.

In some embodiments, the formulations further comprise sucrose. In some embodiments, the formulations have a sucrose concentration of 200 mM to 400 mM. In some embodiments, the formulations have a sucrose concentration of 300 mM.

In some embodiments, the increase in percentage of high molecular weight species in the formulations after storage for one month at 40° C. is less than 4.

In some embodiments, the formulation has a viscosity suitable for injection through a needle or other suitable device in a range of 27 gauge to 31 gauge (e.g., 29 gauge to 31 gauge) in size while at room temperature (for example from 15-30° C., e.g., between 20° C. and 25° C.) or when administered at higher than room temperature, for example in a temperature in range of 40-60° C. In some embodiments, the formulation has a viscosity suitable for injection through a needle or other suitable device having a gauge selected from the group consisting of 27 gauge, 28 gauge, 29 gauge, 30 gauge and 31 gauge, while at room temperature or at a temperature in a range of 40-60° C. In some embodiments, the formulation has a viscosity suitable for injection through a needle or other suitable device having an inner diameter selected from the group consisting of 0.18 mm, 0.16 mm, and 0.14 mm, while at room temperature or at a temperature in a range of 40-60° C. In some embodiments, the formulations have a viscosity of less than 40 cP (e.g., 30 to 39 cP) at anti-CD40 antibody concentrations of up to 250 mg/ml. In some embodiments, the formulations have a viscosity of less than 20 cP at anti-CD40 antibody concentrations of up to 200 mg/ml.

In some embodiments, the formulations comprise 250 mg/ml anti-CD40 antibody, 20 mM citrate, 150 mM arginine, have a pH of 5.5 to 6.1 and have a viscosity of 37 to 39 cP.

In some embodiments, the formulations comprise 250 mg/ml anti-CD40 antibody, 20 mM histidine, 150 mM arginine, have a pH of 5.5 to 6.1 and have a viscosity of 37 to 39 cP.

Some aspects of the present disclosure provide methods producing an anti-CD40 antibody formulation, the method comprising combining 100 mg/ml to 300 mg/ml anti-CD40 antibody in buffer, and adjusting pH of the formulation to 5.0 to 6.5. In some embodiments, the methods comprise combining 100 mg/ml to 300 mg/ml anti-CD40 antibody in citrate buffer, and adjusting pH of the formulation to 5.5 to 6.0. In some embodiments, the methods comprise combining 100 mg/ml to 300 mg/ml anti-CD40 antibody in histidine buffer, and adjusting pH of the formulation to 5.0 to 6.5 (e.g., 5.2-6.2).

Some aspects of present disclosure provide anti-CD40 antibody formulation comprising 100 mg/ml to 300 mg/ml anti-CD40 antibody, 10 mM to 30 mM histidine buffer, 100 mM to 150 mM arginine, 10% to 15% sucrose and 0.05% to 0.10% polysorbate-80.

In some embodiments, anti-CD40 antibody formulations comprise 100 mg/ml to 300 mg/ml anti-CD40 antibody, 20 mM histidine buffer, 100 mM arginine, 5% sucrose and 0.08% polysorbate-80.

### BRIEF DESCRIPTION OF DRAWINGS

FIG. 1 depicts a graph showing total enthalpy (ΔH) values as a function of pH. The maximal ΔH values, indicating maximum folded structure, are attained between a pH value of 5.0 and a pH value of 6.0.

FIG. 2 depicts a graph showing an increase in the percentage (%) of high molecular weight (HMW) species formed—indicative of protein aggregates—after storage for 1 month at 40° C. in antibody formulations containing 20 mM citrate, 150 mM arginine (Arg) and 150 mg/ml, 200 mg/ml or 250 mg/ml anti-CD40 antibody and having a pH value of approximately 5.7. There is a minimal increase in aggregation under these conditions.

FIGS. 3A and 3B depict graphs showing an increase in the percentage (%) of HMW species formed after storage for 1 month at 40° C. in two different antibody formulations: 150 mg/ml, 200 mg/ml or 250 mg/ml anti-CD40 antibody in 20 mM citrate (FIG. 3A), and 150 mg/ml, 200 mg/ml or 250 mg/ml anti-CD40 antibody in 20 mM citrate, 300 mM sucrose (FIG. 3B)—trends are similar to those show in FIG. 2.

FIGS. 4A-4C depict graphs showing viscosity as a function of pH in different antibody formulations: 150 mg/ml, 200 mg/ml or 250 mg/ml anti-CD40 antibody in 20 mM citrate, 150 mM Arg (FIG. 4A); 150 mg/ml, 200 mg/ml or 250 mg/ml anti-CD40 antibody in 20 mM citrate (FIG. 4B); and 150 mg/ml, 200 mg/ml or 250 mg/ml anti-CD40 antibody in 20 mM citrate, 300 mM sucrose. Note that viscosity for 250 mg/ml CD40 is shown on the right-hand Y-axis. Similar to the data shown in FIGS. 2 and 3A-3B, the viscosity of different formulations with different protein concentrations tends to a minimum at a pH value of approximately 5.7.

FIG. 5A depicts a graph showing the effect of arginine concentration on viscosity of anti-CD40 antibody solutions. 250 mg/ml viscosity data is charted on right axis. FIGS. 5B-5C depict graphs showing an increase in aggregation at each time point following incubation at 40° C.

FIGS. 6A-6C depict graphs showing the effect of arginine, sucrose and no excipients on anti-CD40 antibody in 20 mM histidine buffer at various pH values. 250 mg/ml viscosity data is charted on right axis.

FIGS. 7A-7C depict graphs showing an increase in aggregation after 1 month at 40° C. for anti-CD40 antibody in 20 mM histidine buffer at various pH and with various excipients.

### DETAILED DESCRIPTION OF INVENTION

Anti-CD40 antibody formulations, as provided herein, have a high antibody concentration, low viscosity, and low aggregation propensity such that they are suitable for subcutaneous injections as well as for other routes of administration. Anti-CD40 antibody formulations of the present disclosure are based on unexpected results showing that both the viscosity and protein aggregation for a formulation containing an anti-CD40 antibody concentration of 100 mg/ml to 300 mg/ml are at lowest values within a narrow range of pH values—pH 5.0 to pH 6.5. In some embodiments, anti-CD40 antibody formulations, as provided herein, also contain citrate buffer (e.g., 10 mM to 30 mM) and/or arginine (e.g., 140 mM to 160 mM), or histidine buffer (e.g., 10 mM to 30 mM) and/or arginine (e.g., 140 mM to 160 mM). Reduced viscosity antibody formulations are particular important for high-concentration formulations that are delivered to a patient via an intravenous or subcutaneous route.

CD40 is a Type 1 transmembrane receptor expressed by B cells, macrophages, dendritic cells, and other cell types, including platelets, epithelial, endothelial, and stromal cells. The engagement of CD40 by its ligand, CD40 ligand (CD40L also known as CD154), constitutes a key axis for the activation of innate and adaptive immune functions. This notably includes B cell functions of clonal expansion, differentiation to antibody forming cells and memory cells expressing isotype-switched antibodies, and the germinal center reaction. Thus, CD40/CD40L is a premier immunological pathway that affects processes thought to be involved in diseases of autoimmunity and humoral immunity (Burkly, Adv. Exp. Med. Biol., 489:135-52 (2001); van Kooten et al., J. Leuk. Biol., 67:2-17 (2000)). Therefore, antibodies that modulate the CD40/CD40L interaction are of interest in treating diseases such as autoimmune and inflammatory diseases.

Blocking CD40 can potentially reduce the above downstream effects of CD40 signaling, dampening the hyperactivation of adaptive and innate immune responses in patients, for example, with autoimmune and inflammatory diseases.

The amino acid sequence of the human CD40 protein (Genbank® Accession No. NP_001241) is shown below (the extracellular domain—P20 to R193—is underlined). Anti-CD40 antibody formulations of the present disclosure may include anti-CD40 antibodies prepared using the human CD40 protein as an immunogen.

| (SEQ ID NO: 1) | |
|---|---|
| 1 | MVRLPLQCVL WGCLLTAVHP EPPTACREKQ YLINSQCCSL CQPGQKLVSD CTEFTETECL |
| 61 | PCGESEFLDT WNRETHCHQH KYCDPNLGLR VQQKGTSETD TICTCEEGWH CTSEACESCV |
| 121 | LHRSCSPGFG VKQIATGVSD TICEPCPVGF FSNVSSAFEK CHPWTSCETK DLVVQQAGTN |
| 181 | KTDVVCGPQD RLRALVVIPI IFGILFAILL VLVFIKKVAK KPTNKAPHPK QEPQEINFPD |
| 241 | DLPGSNTAAP VQETLHGCQP VTQEDGKESR ISVQERQ |

The amino acid sequence of cynomolgus CD40 protein (Genbank Accession No. XP_005569275) is shown below. Cysteine rich domain 1 (CRD1) is in bold text; CRD2 is underlined; CRD3 is italicized; and CRD4 is both in bold text and underlined. The cynomolgus CD40 protein is 93% identical to the human CD40 protein. Anti-CD40 antibody formulations of the present disclosure may include anti-CD40 antibodies prepared using the cynomolgus CD40 protein as an immunogen.

| (SEQ ID NO: 2) | |
|---|---|
| 1 | MVRLPLQCVL WGCLLTAVYP EPPTACREKQ YLINSQCCSL CQPGQKLVSD CTEFTETECL |
| 61 | PCGESEFLDT WNRETRCHQH KYCDPNLGLR VQQKGTSETD TICTCEEGLH CTSESCESCV |
| 121 | PHRSCLPGFG VKQIATGVSD TICEPCPVGF FSNVSSAFEK CRPWTSCETK DLVVQQAGTN |
| 181 | KTDVVCGPQD RQRALVVIPI CLGILFVILL LVLVFIKKVA KKPNDKVPHP KQEPQEINFP |
| 241 | DDLPGSNPAA PVQETLHGCQ PVTQEDGKES RISVQERQ |

Anti-CD40 antibody formulations of the present disclosure, in some embodiments, contain anti-CD40 antibodies that can block the CD40/CD40L interaction and thus are useful in treating immunological diseases such as autoimmune disorders and inflammatory disorders. These antibodies all bind human CD40. Such anti-CD40 antibodies include, without limitation, anti-CD40 monoclonal antibodies, such as a humanized IgG4/kappa monoclonal antibody with serine at position 225 (S228 according to Kabat numbering) of the heavy chain hinge region changed to proline to avoid half antibody formation in vivo (IgG4P).

In some embodiments, an anti-CD40 antibody of the present disclosure includes the amino acid sequences shown below. Complementarity-determining regions (CDRs) 1, 2, and 3, according to Kabat numbering, of the variable light chain (VL) and the variable heavy chain (VH) are shown in that order from the N to the C-terminus of the mature VL and VH sequences and are both underlined and in bold.

### Example Anti-CD40 Antibody—Light Chain

| (SEQ ID NO: 3) | |
|---|---|
| 1 | DIQMTQSPSS LSASVGDRVT ISCRASQDIS NYLNWYQQKP GKVPKLLIYF |
| 51 | TSRLRSGVPS RFSGSGSGTD YTLTISSLQP EDVATYYCQQ DRKLPWTFGQ |
| 101 | GTKLEIKRTV AAPSVFIFPP SDEQLKSGTA SVVCLLNNFY PREAKVQWKV |
| 151 | DNALQSGNSQ ESVTEQDSKD STYSLSSTLT LSKADYEKHK VYACEVTHQG |
| 201 | LSSPVTKSFN RGEC |

### Example Anti-CD40 Antibody—Heavy Chain

| (SEQ ID NO: 4) | |
|---|---|
| 1 | EVQLVQSGAE VKKPGASVKV SCKASGYTFT TFPIEWVRQA PGQGLEWMGN |
| 51 | FHPYNDDTKY NEKFKGRVTL TADKSTSTAY MELSRLRSED TAVYYCARRG |
| 101 | KLPFDSWGQG TTVTVSSAST KGPSVFPLAP CSRSTSESTA ALGCLVKDYF |
| 151 | PEPVTVSWNS GALTSGVHTF PAVLQSSGLY SLSSVVTVPS SSLGTKTYTC |
| 201 | NVDHKPSNTK VDKRVESKYG PPCPPCPAPE FLGGPSVFLF PPKPKDTLMI |
| 251 | SRTPEVTCVV VDVSQEDPEV QFNWYVDGVE VHNAKTKPRE EQFNSTYRVV |
| 301 | SVLTVLHQDW LNGKEYKCKV SNKGLPSSIE KTISKAKGQP REPQVYTLPP |
| 351 | SQEEMTKNQV SLTCLVKGFY PSDIAVEWES NGQPENNYKT TPPVLDSDGS |
| 401 | FFLYSRLTVD KSRWQEGNVF SCSVMHEALH NHYTQKSLSL SLG |

In some embodiments, the VL of an anti-CD40 antibody has the following amino acid sequence:

| (SEQ ID NO: 5) | |
|---|---|
| 1 | DIQMTQSPSS LSASVGDRVT ISCRASQDIS NYLNWYQQKP GKVPKLLIYF |
| 51 | TSRLRSGVPS RFSGSGSGTD YTLTISSLQP EDVATYYCQQ DRKLPWTFGQ |
| 101 | GTKLEIK |

In some embodiments, the VH of an anti-CD40 antibody has the following amino acid sequence:

| (SEQ ID NO: 6) | |
|---|---|
| 1 | EVQLVQSGAE VKKPGASVKV SCKASGYTFT TFPIEWVRQA PGQGLEWMGN |
| 51 | FHPYNDDTKY NEKFKGRVTL TADKSTSTAY MELSRLRSED TAVYYCARRG |
| 101 | KLPFDSWGQG TTVTVSS |

In some embodiments, the amino acid sequences of VL CDRs (according to Kabat) of an anti-CD40 antibody comprise/consist of the following amino acid sequence:

VL CDR1:

(SEQ ID NO: 7)

RASQDISNYLN;
VL CDR2:

(SEQ ID NO: 8)

FTSRLRS;
and
VL CDR3:

(SEQ ID NO: 9)

QQDRKLPWT.

In some embodiments, the amino acid sequences of the VH CDRs (according to Kabat) of an anti-CD40 antibody comprise/consist of the following amino acid sequence:

VH CDR1:

(SEQ ID NO: 10)

TFPIE;
VH CDR2:

(SEQ ID NO: 11)

NFHPYNDDTKYNEKFKG;
and
VH CDR3:

(SEQ ID NO: 12)

RGKLPFDS.

Anti-CD40 antibodies, or antigen binding fragments thereof, of the present disclosure can encompass the heavy chain CDR 1, CDR2, and CDR3 and the light chain CDR 1, CDR2, and CDR3 of the example anti-CD40 antibody described above. In some embodiments, anti-CD40 antibodies, or antigen binding fragments thereof, comprise an amino acid sequence having at least 80%, 85%, 86%, 87%, 88%, 89%, 90%, 91%, 92%, 93%, 94%, 95%, 96%, 97%, 98%, 99%, or 100% identity to the variable heavy chain and/or the variable light chain of the example anti-CD40 antibody described above.

Anti-CD40 antibodies of the present disclosure include, without limitation, monoclonal antibodies, polyclonal antibodies, chimeric antibodies, humanized antibodies, and fragments thereof. Other anti-CD40 antibodies are contemplated herein.

Aspects of the present disclosure provide high-concentration anti-CD40 antibody formulations. An anti-CD40 antibody “formulation” refers simply to a mixture of components that includes an anti-CD40 antibody. A formulation may be a solution (e.g., an aqueous solution) or it may be in lyophilized form (e.g., and later reconstituted in buffer or water for administration). An anti-CD40 antibody formulation may comprise, consist of, or consist essentially of anti-CD40 antibody, citrate, arginine, sucrose, polysorbate or any combination of two or more of the foregoing. An anti-CD40 antibody formulation may comprise, consist of, or consist essentially of anti-CD40 antibody, histidine, arginine, sucrose, polysorbate or any combination of two or more of the foregoing.

Anti-CD40 antibody formulations of the present disclosure have a low viscosity such that they are suitable for subcutaneous injections as well as for other routes of administration. “Viscosity” of a fluid is a measure of its resistance to gradual deformation by shear stress or tensile stress. For liquids, viscosity corresponds to the informal concept of being of a thick, sticky and semifluid consistency. Viscosity is often measured by a viscometer and expressed in centipoise (cP) units.

Anti-CD40 antibody formulations of the present disclosure have a low protein aggregation propensity such that they are suitable for subcutaneous injections as well as for other routes of administration. Protein aggregation is a biological phenomenon in which misfolded proteins aggregate (e.g., accumulate and clump together) either intracellularly or extracellularly. Protein aggregates are often toxic. In some embodiments, the anti-CD40 antibody aggregation is reduced by at least 10%, 20%, 30%, 40%, 50%, 60%, 70%, or at least 80% relative to other anti-CD40 antibody formulations (e.g., having comparable anti-CD40 antibody concentrations formulated in non-citrate buffer or at a pH value of less than 5.0 or greater than 6.5). In some embodiments, the anti-CD40 antibody aggregation is reduced by at least 10%, 20%, 30%, 40%, 50%, 60%, 70%, or at least 80%. In some embodiments, an anti-CD40 antibody formulation has reduced antibody aggregation when stored at 5° C. to 40° C. (e.g., 5° C., 25° C. and/or 40° C.). Methods of assessing antibody aggregation are familiar to one of ordinary skill in the art and include, without limitation, methods such as protein gel electrophoresis and protein staining, Western blotting, mass spectrometry and size-exclusion chromatography. Other methods of assessing antibody aggregation are contemplated herein.

For an antibody formulation to be useful for subcutaneous injection, its viscosity must be low enough for the formulation to flow easily through a fine gauge needle without requiring an excessively high force. In some embodiments, anti-CD40 antibody formulations, as provided herein, have a viscosity suitable for subcutaneous injection. In some embodiments, the viscosity of an antibody formulation is less than 100 centipoise (cP). In some embodiments, the viscosity of an antibody formulation is between 1 cP and 100 cP. In some embodiments, the viscosity of an antibody formulation is 10 cP to 50 cP. In some embodiments, the viscosity of an antibody formulation is 15 cP to 35 cP. In some embodiments, the viscosity of an antibody formulation is 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP or 40 cP. In some embodiments, an antibody formulation has a viscosity suitable for injection through a needle having a size of 29 gauge to 31 gauge. In some embodiments, an antibody formulation has a viscosity suitable for injection through a needle having a size of 29 gauge to 31 gauge while at a temperature of 25° C. to 40° C. In some embodiments, formulation has a viscosity of less than 40 cP at anti-CD40 antibody concentrations of up to 250 mg/ml. In some embodiments, the formulation has a viscosity of up to 20 cP at anti-CD40 antibody concentrations of up to 200 mg/ml.

In some embodiments, the concentration of an anti-CD40 antibody formulation is at least 100 mg/ml. For example, the concentration of an anti-CD40 antibody formulation may be at least 150 mg/ml, 200 mg/ml, 250 mg/ml or 300 mg/ml. In some embodiments, the concentration of an anti-CD40 antibody formulation is 100 mg/ml to 300 mg/ml. For example, the concentration of an anti-CD40 antibody formulation may be 100 mg/ml, 105 mg/ml, 110 mg/ml, 115 mg/ml, 120 mg/ml, 125 mg/ml, 130 mg/ml, 135 mg/ml, 140 mg/ml, 145 mg/ml, 155 mg/ml, 160 mg/ml, 165 mg/ml, 170 mg/ml, 175 mg/ml, 180 mg/ml, 185 mg/ml, 190 mg/ml, 195 mg/ml, 200 mg/ml, 205 mg/ml, 210 mg/ml, 215 mg/ml, 220 mg/ml, 225 mg/ml, 230 mg/ml, 235 mg/ml, 240 mg/ml, 245 mg/ml, 255 mg/ml, 260 mg/ml, 265 mg/ml, 270 mg/ml, 275 mg/ml, 280 mg/ml, 285 mg/ml, 290 mg/ml, 295 mg/ml or 300 mg/ml. In some embodiments, the concentration of an anti-CD40 antibody formulation is 150 mg/ml to 250 mg/ml.

The pH value of anti-CD40 antibody formulations of the present disclosure may be 5.7±0.5 (e.g., 5.2, 5.3, 5.4, 5.5, 5.6, 5.7, 5.8, 5.9, 6.0 or 6.1). In some embodiments, the pH value of anti-CD40 antibody formulation is 5.5. to 6.0, 5.5 to 5.9, 5.5 to 5.8, 5.6 to 6.0, 5.6 to 5.9 or 5.6 to 5.8. In some embodiments, the pH value of anti-CD40 antibody formulation is greater than 5.5. In some embodiments, the pH value of anti-CD40 antibody formulation is less than 6.0. In some embodiments, the pH value of anti-CD40 antibody formulation is greater than 5.5 and less than 6.0. In some embodiments, the pH value of anti-CD40 antibody formulation is 5.7.

In some embodiments, anti-CD40 antibody formulations of the present disclosure contains citrate. Citrate is a derivative of citric acid. Citrate may be in the form of a salt, and ester or a polyatomic anion. For example, a salt may be a sodium citrate (e.g., a mixture of mono-, di- and/or trisodium) a potassium citrate, and ammonium citrate, and an ester may be an ethyl citrate. The concentration of citrate in an anti-CD40 antibody formulation, as provided herein, may be 5 mM to 50 mM. For example, the concentration of citrate in an anti-CD40 antibody formulation may be 5 mM, 10 mM, 15 mM, 20 mM, 25 mM, 30 mM, 35 mM, 40 mM, 45 mM or 50 mM. In some embodiments, the concentration of citrate in an anti-CD40 antibody formulation is 10 mM to 30 mM. In some embodiments, the concentration of citrate in an anti-CD40 antibody formulation is at least 20 mM. In some embodiments, the concentration of citrate in an anti-CD40 antibody formulation is 20 mM. In some embodiments, the concentration of citrate in an anti-CD40 antibody formulation is less than 5 mM or greater than 500 mM.

In some embodiments, anti-CD40 antibody formulations of the present disclosure contains histidine (His) (e.g., in the form of a histidine buffer). Histidine is an α-amino acid that is used in the biosynthesis of proteins. It contains an α-amino group, a carboxylic acid group, and a side chain imidazole, classifying it as a positively charged (at physiological pH), aromatic amino acid. The concentration of histidine in an anti-CD40 antibody formulation, as provided herein, may be 5 mM to 50 mM. For example, the concentration of histidine in an anti-CD40 antibody formulation may be 5 mM, 10 mM, 15 mM, 20 mM, 25 mM, 30 mM, 35 mM, 40 mM, 45 mM or 50 mM. In some embodiments, the concentration of histidine in an anti-CD40 antibody formulation is 10 mM to 30 mM. In some embodiments, the concentration of histidine in an anti-CD40 antibody formulation is at least 20 mM. In some embodiments, the concentration of histidine in an anti-CD40 antibody formulation is 20 mM. In some embodiments, the concentration of histidine in an anti-CD40 antibody formulation is less than 5 mM or greater than 500 mM.

In some embodiments, an anti-CD40 antibody formulation contains or is formulated using “citrate buffer.” In some embodiments, citrate buffer may contain sodium citrate and citric acid. In some embodiments, a citrate buffer may contain sodium phosphate and citric acid. A “buffer” refers to a pH-controlled liquid that can be used to dissolve an active ingredient (e.g., pharmaceutically active ingredient) and/or salts and excipients (e.g., pharmaceutically acceptable salts and excipients), or as a diluent to dissolve liquid compounds and compositions. Buffers neutralize small amounts of acid or base present within a solution and maintain a stable pH of the solution. In some embodiments, buffers comprise weak acid-base conjugate pair. Non-limiting examples of compounds comprising weak acid-base conjugate pairs include, without limitation, maleic acid, tartaric acid, lactic acid, citric acid, acetic acid, sodium bicarbonate, sodium phosphate and tris(hydroxymethyl)aminomethane (Tris/Tram).

In some embodiments, anti-CD40 antibody formulations of the present disclosure contain arginine. The concentration of arginine in an anti-CD40 antibody formulation, as provided herein, may be 100 mM to 200 mM. For example, the concentration of arginine in an anti-CD40 antibody formulation may be 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM or 200 mM. In some embodiments, the concentration of arginine in an anti-CD40 antibody formulation is 140 mM to 160 mM. In some embodiments, the concentration of arginine in an anti-CD40 antibody formulation is at least 150 mM. In some embodiments, the concentration of arginine in an anti-CD40 antibody formulation is 150 mM. In some embodiments, the concentration of arginine in an anti-CD40 antibody formulation is less than 100 mM or greater than 200 mM.

In some embodiments, anti-CD40 antibody formulations of the present disclosure contain histidine. The concentration of histidine in an anti-CD40 antibody formulation, as provided herein, may be 100 mM to 200 mM. For example, the concentration of histidine in an anti-CD40 antibody formulation may be 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM or 200 mM. In some embodiments, the concentration of histidine in an anti-CD40 antibody formulation is 140 mM to 160 mM. In some embodiments, the concentration of histidine in an anti-CD40 antibody formulation is at least 150 mM. In some embodiments, the concentration of histidine in an anti-CD40 antibody formulation is 150 mM. In some embodiments, the concentration of histidine in an anti-CD40 antibody formulation is less than 100 mM or greater than 200 mM.

In some embodiments, anti-CD40 antibody formulations of the present disclosure contain lysine. The concentration of lysine in an anti-CD40 antibody formulation, as provided herein, may be 100 mM to 200 mM. For example, the concentration of lysine in an anti-CD40 antibody formulation may be 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM or 200 mM. In some embodiments, the concentration of lysine in an anti-CD40 antibody formulation is 140 mM to 160 mM. In some embodiments, the concentration of lysine in an anti-CD40 antibody formulation is at least 150 mM. In some embodiments, the concentration of lysine in an anti-CD40 antibody formulation is 150 mM. In some embodiments, the concentration of lysine in an anti-CD40 antibody formulation is less than 100 mM or greater than 200 mM.

In some embodiments, anti-CD40 antibody formulations of the present disclosure contain one or more of methionine, aspartic acid, glycine, and glutamic acid, for example, at a concentration of 100 mM to 200 mM.

In some embodiments, anti-CD40 antibody formulations of the present disclosure include a sugar (e.g., sucrose, trehalose, mannitol, sorbitol, or xylitol) and/or a tonicity modifier (e.g., mannitol, or sorbitol) and/or a surfactant (e.g., polysorbate-20 or polysorbate-80).

In some embodiments, anti-CD40 antibody formulations of the present disclosure contain sucrose. The concentration of sucrose in an anti-CD40 antibody formulation, as provided herein, may be 100 mM to 500 mM. For example, the concentration of sucrose in an anti-CD40 antibody formulation may be 100 mM, 110 mM, 120 mM, 130 mM, 140 mM, 150 mM, 160 mM, 170 mM, 180 mM, 190 mM, 200 mM, 220 mM, 220 mM, 230 mM, 240 mM, 250 mM, 260 mM, 270 mM, 280 mM, 290 mM, 300 mM, 330 mM, 320 mM, 330 mM, 340 mM, 350 mM, 360 mM, 370 mM, 380 mM, 390 mM, 400 mM, 410 mM, 420 mM, 440 mM, 440 mM, 450 mM, 460 mM, 470 mM, 480 mM, 490 mM or 500 mM. In some embodiments, the concentration of sucrose in an anti-CD40 antibody formulation is 200 mM to 350 mM. In some embodiments, the concentration of sucrose in an anti-CD40 antibody formulation is 300 mM. In some embodiments, the concentration of sucrose in an anti-CD40 antibody formulation is less than 100 mM or greater than 500 mM. In some embodiments, the concentration of sucrose in an anti-CD40 antibody formulation is at least 5%. For example, the concentration of sucrose in an anti-CD40 antibody formulation may be 5%, 6%, 7%, 8%, 9%, 10%, 11%, 12%, 13%, 14%, 15%, 16%, 17%, 18%, 19% or 20%. In some embodiments, the concentration of sucrose in an anti-CD40 antibody formulation is less than 5% or greater than 20%.

In some embodiments, anti-CD40 antibody formulations of the present disclosure contain other excipients such as succinate, methionine, glycine, sorbitol, or polysorbate-80 (Tween-80). In some embodiments, the concentration of excipient (e.g., polysorbate 80) in an anti-CD40 antibody formulation is at least 0.05%. For example, the concentration of excipient (e.g., polysorbate 80) in an anti-CD40 antibody formulation may be 0.05%, 0.06%, 0.07%, 0.08%, 0.09% or 0.1%. In some embodiments, the concentration of excipient in an anti-CD40 antibody formulation is less than 0.05% or greater than 0.1%.

In some embodiments, anti-CD40 antibody compositions or formulations described herein are administered to a subject (e.g., subcutaneously). A subject can be a vertebrate, mammal, including but not limited to, a human, a non-human primate, a rodent, an ovine, a bovine, or other mammal. A subject can be a subject in need of treatment with an anti-CD40 antibody. In some embodiments a small volume of an anti-CD40 antibody liquid formulation (e.g., 0.5-2.0 mL, or around 1 mL) containing a relatively high concentration of anti-CD40 antibody (e.g., over 50 mg/mL, around 100 mg/mL or more, 100-150 mg/mL, around 150 mg/mL, 150-200 mg/mL, around 200 mg/mL, 200-250 mg/mL, around 250 mg/mL, 250-350 mg/mL, 350-450 mg/mL, or more) having a viscosity of less than 100 cP (e.g., less than 50 cP) is administered to a subject. It should be appreciated that in some embodiments, formulations described herein can be used for small volume administration (e.g., between 0.5 mL and 2 mL, for example around 1 mL injection volumes). It also should be appreciated that in some embodiments, one or more formulations described herein can be sterilized using any suitable technique (e.g., filtration or other technique) in order to produce a composition that is suitable (e.g., sterile) for administration to a subject.

Accordingly, anti-CD40 antibody formulations of the present disclosure can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

Anti-CD40 antibody formulations, as provided herein, can be administered as a fixed dose, or mg/kg (of the subject body weight) dose. The dose can also be chosen to reduce or avoid production of antibodies against the anti-CD40 antibody. Dosage regimens are adjusted to provide the desired response, e.g., a therapeutic response or a combinatorial therapeutic effect. In some embodiments, doses of the anti-CD40 antibody formulation (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In some embodiments, a subject in need of treatment with an anti-CD40 antibody formulation of the present disclosure is administered the antibody at a dose 2 mg/kg, 4 mg/kg, 5 mg/kg, 10 mg/kg, 15 mg/kg, 20 mg/kg, 30 mg/kg, 35 mg/kg, or 40 mg/kg.

Dosage unit form or “fixed dose” refers to physically discrete units suited as unitary dosages for the subjects to be treated; each unit contains a predetermined quantity of anti-CD40 antibody calculated to produce the desired therapeutic effect in association with the required pharmaceutical carrier and optionally in association with the other agent. Single or multiple dosages may be given. Alternatively, or in addition, anti-CD40 antibody formulation, as provided herein, may be administered via continuous infusion.

In some embodiments, anti-CD40 antibody formulations are formulated for subcutaneous injection. Thus, anti-CD40 antibody formulations, as provided herein, may be administered subcutaneously (via injection) at concentrations of up to 300 mg/ml (e.g., 100 mg/ml to 300 mg/ml) in a volume of 1 ml or less. In some embodiments, a formulation of the present disclosure has an anti-CD40 antibody concentration of 100 mg/ml, 125 mg/ml, 150 mg/ml, 175 mg/ml, 200 mg/ml, 225 mg/ml, 250 mg/ml, 275 mg/ml or 300 mg/ml in a volume of 0.5 ml, 0.55 ml, 0.6 ml, 0.65 ml, 0.7 ml, 0.75 ml, 0.8 ml, 0.85 ml, 0.9 ml, 0.95 ml or 1.0 ml. In some embodiments, the volume of anti-CD40 antibody formulation is less than 0.5 ml or greater than 1.0 ml.

An anti-CD40 antibody formulation dose can be administered, for example, at a periodic interval over a period of time (a course of treatment) sufficient to encompass at least 2 doses, 3 doses, 5 doses, 10 doses, or more, e.g., once or twice daily, or one to four times per week, or weekly, biweekly (every two weeks), every three weeks, monthly, e.g., for 1 to 12 weeks, 2 to 8 weeks, 3 to 7 weeks, or for 4, 5, or 6 weeks. In some embodiments, an anti-CD40 antibody formulation is administered biweekly. In a some embodiments, an anti-CD40 antibody formulation is administered monthly. Factors that may influence the dosage and timing required to effectively treat a subject include, for example, the severity of the disease or disorder, formulation, route of delivery, previous treatments, the general health and/or age of the subject, and other diseases present. Moreover, treatment of a subject with a therapeutically effective amount of an anti-CD40 antibody formulation of the present disclosure can include a single treatment or, preferably, can include a series of treatments.

If a subject is at risk for developing an immunological disorder described herein, the an anti-CD40 antibody formulation can be administered, in some embodiments, before the full onset of the immunological disorder, e.g., as a preventative measure. The duration of such preventative treatment can be a single dosage of the anti-CD40 antibody formulation or the treatment may continue (e.g., multiple dosages). For example, a subject at risk for the disorder or who has a predisposition for the disorder may be treated with the anti-CD40 antibody formulation for days, weeks, months, or even years so as to prevent the disorder from occurring or fulminating.

An anti-CD40 antibody formulation may be referred to as a “pharmaceutical formulation.” Typically, a pharmaceutical formulation includes a pharmaceutically acceptable carrier. A “pharmaceutically acceptable carrier” includes any and all solvents, dispersion media, coatings, antibacterial and antifungal agents, isotonic and absorption delaying agents, and the like that are physiologically compatible. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

An anti-CD40 antibody formulation, or a pharmaceutical formulation, may include a “therapeutically effective amount” of an anti-CD40 antibody. Such effective amounts can be determined based on the effect of the anti-CD40 antibody, or the combinatorial effect of anti-CD40 antibody if a second agent is used. A therapeutically effective amount of anti-CD40 antibody may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the anti-CD40 antibody formulation are outweighed by the therapeutically beneficial effects.

Anti-CD40 antibody formulations, as provided herein, can be used, in some embodiments, to treat or prevent a variety of immunological disorders, such as autoimmune disorders, inflammatory diseases, disorders of humoral immunity, and fibrotic disorders. In addition, anti-CD40 antibody formulations are also useful in treating antibody-mediated diseases as well as neurological disorders. Anti-CD40 antibody formulations are useful to treat or prevent such disorders at least because they inhibit or block the interaction of CD40 with its ligand, CD40L (CD154). CD40 signaling constitutes an important component in the activation of innate and adaptive immune functions, notably including B cell clonal expansion, differentiation to antibody forming cells (AFC) and memory cells expressing isotype-switched antibodies, the germinal center (GC) reaction, and optimal T helper effector cell responses.

The term “treating” refers to administering an anti-CD40 formulation of the present disclosure in an amount, manner, and/or mode effective to improve a condition, symptom, or parameter associated with a disorder or to prevent progression or exacerbation of the disorder (including secondary damage caused by the disorder) to either a statistically significant degree or to a degree detectable to one skilled in the art.

Autoimmune diseases that can be treated or prevented with formulations as provided herein include, without limitation, Sjögren's syndrome (e.g., primary Sjögren's syndrome (pSS)), SLE (e.g., moderate or severe lupus), lupus nephritis, cutaneous lupus, discoid lupus, systemic sclerosis (scleroderma), acquired hemophilia, Crohn's disease, ulcerative colitis, Graves disease, Idiopathic thrombocytopenic purpura (ITP), rheumatoid arthritis (RA), asthma, vasculitis, pemphigoid, atopic dermatitis, and hemolytic anemia.

Antibody-mediated diseases or conditions where the formulations, as provided herein, are useful include, without limitation, hemophilia with inhibitors, transplant rejection, antibody cross-match pre-transplant, alloantibody in transfusion, and graft versus host disease.

Neurological diseases that can be treated or prevented with formulations as provided herein include, without limitation, myasthenia gravis, Alzheimer's disease, neuromyelitis optica (NMO), and Amyotrophic lateral sclerosis (ALS).

### EXAMPLES

### Example 1

Anti-CD40 antibody was dialyzed against 20 mM citrate buffer at pH values of 2.0, 3.0, 4.0, 5.0, 6.0, 7.0, 8.0, 9.0 and 10.0 and subsequently diluted to 1 mg/ml. Aliquots of 400 μl of anti-CD40 antibody formulation were placed in deep-well plates in the sample compartment of a Microcal VP-Capillary DSC. The samples were heated from 10° C. to 110° C. at 100° C./h. Data were baseline-subtracted and fitted to a non-two-state model to extract the individual Tₘ-values and ΔH, enthalpies of unfolding. The latter values were summed to determine the total enthalpy of unfolding. The Tₘ values are shown in Table 1. All Tₘ values are lower at a pH value of less than 5.0. At pH values of 5.0 and greater, the anti-CD40 antibody exhibited conformational stability. Based on conformational stability alone, any pH value above 5.0 would be suitable for formulation.

TABLE 1
Thermal stability at different pH values.

| pH | Tₘ1 (° C.) | Tₘ2 (° C.) | Tₘ3 (° C.) | Tₘ4 (° C.) |
|---|---|---|---|---|
| 2 | 47.84 ± 0.49 | 56.63 ± 0.28 | 60.45 ± 0.09 | |
| 3 | 58.22 ± 0.01 | 66.01 ± 0.04 | 72.27 ± 0.24 | |
| 4 | 51.47 ± 0.09 | 58.50 ± 0.20 | 71.01 ± 0.36 | 73.83 ± 0.10 |
| 5 | 61.46 ± 0.07 | 70.36 ± 0.14 | 79.12 ± 0.34 | 81.83 ± 0.10 |
| 6 | 67.27 ± 0.23 | 71.90 ± 0.77 | 80.67 ± 0.33 | 83.46 ± 0.12 |
| 7 | 67.35 ± 0.03 | 77.12 ± 0.25 | 79.59 ± 0.09 | |
| 8 | 66.80 ± 0.03 | 76.83 ± 0.25 | 79.32 ± 0.09 | |
| 9 | 67.35 ± 0.03 | 77.12 ± 0.25 | 79.59 ± 0.09 | |
| 10 | 66.80 ± 0.03 | 76.83 ± 0.25 | 79.32 ± 0.09 | |

### Example 2

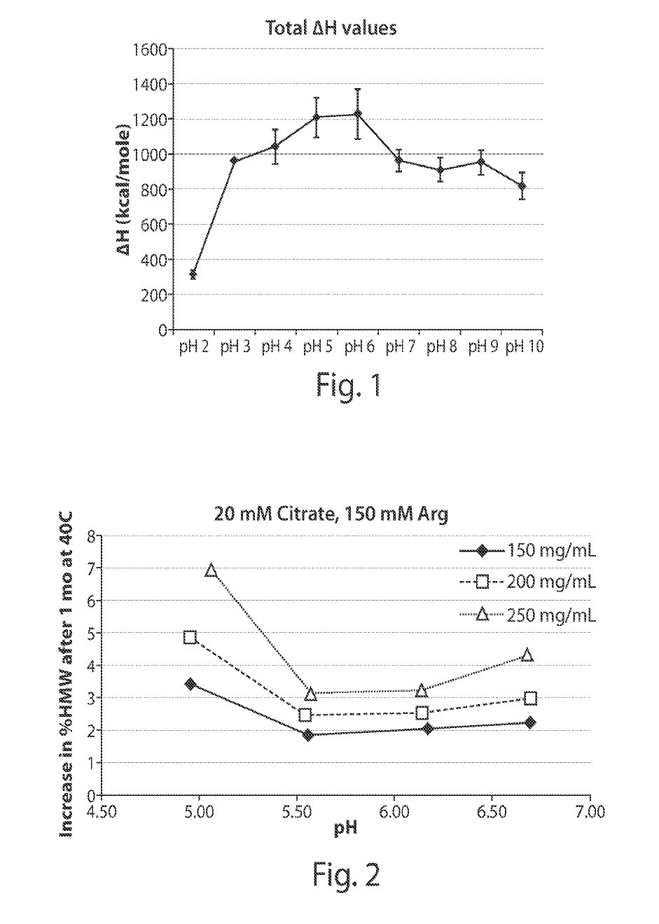

Anti-CD40 antibody was dialyzed against 20 mM citrate buffer at pH values of 2.0, 3.0, 4.0, 5.0, 6.0, 7.0, 8.0, 9.0 and 10.0 and subsequently diluted to 1 mg/ml of 20 mM citrate buffer. Aliquots of 400 μl of anti-CD40 antibody formulation were placed in deep-well plates in the sample compartment of a Microcal VP-Capillary DSC. The samples were heated from 10° C. to 110° C. at 100° C./h. Data were baseline-subtracted and fitted to a non-two state model to extract the individual Tₘ-values and ΔH, enthalpies of unfolding. The latter values were summed to determine the total enthalpy of unfolding. The ΔHₜₒₜₐₗ values are shown in FIG. 1. The ΔHₜₒₜₐₗ is an indication of the extent of antibody structure. The ΔHₜₒₜₐₗ is minimized at the extremes of pH (e.g., pH 1-3, pH 9-10), while maximized at pH 5.0-6.0. This suggests that at pH values of 5.0-6.0, maximal antibody folding occurs.

### Example 3

Anti-CD40 antibody was dialyzed into each formulation (20 mM citrate buffer at pH values of 5.0, 5.7, 6.4 or 7.0) with 150 mM arginine. After dialysis, each antibody formulation was concentrated (e.g., to 150 mg/ml, 200 mg/ml or 250 mg/ml). The pH of samples of the concentrated formulations was measured, the samples were incubated at 40° C., and the percentage (%) of high molecular weight (HMW) species was measured at several different times following incubation. The increase in protein aggregation as reflected by the percentage of HMW species after a one-month incubation is shown in FIG. 2. At each concentration (e.g., to 150 mg/ml, 200 mg/ml or 250 mg/ml), the increase in protein aggregation was minimized in the pH range of pH 5.5-6.1.

### Example 4

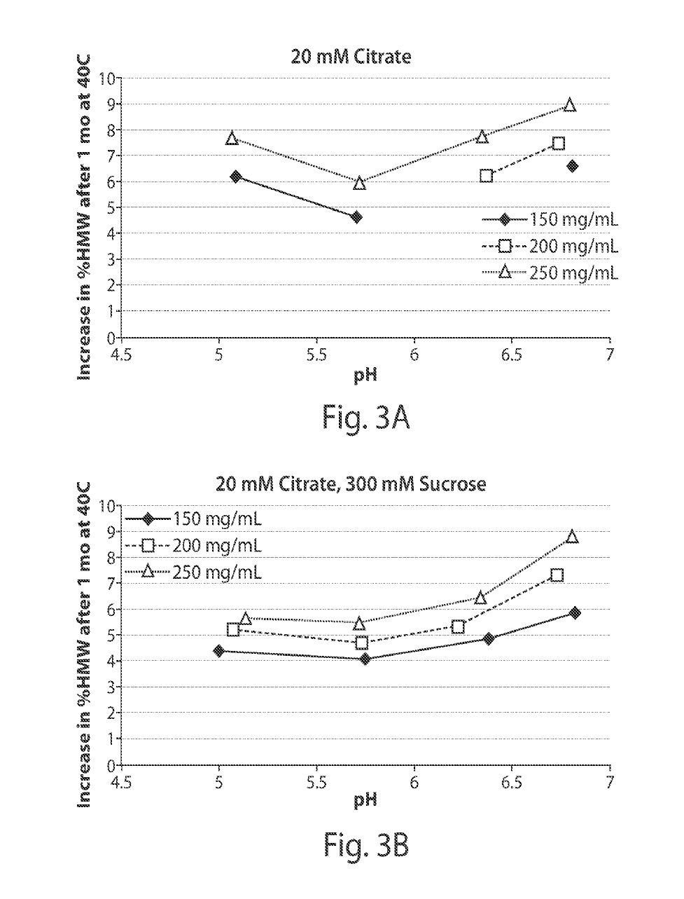

Anti-CD40 antibody was dialyzed into each formulation 20 mM citrate at pH values of 5.0, 5.7, 6.4 or 7.0, with or without the presence of 300 mM sucrose. Following dialysis, each formulation was concentrated (e.g., to 150 mg/ml, 200 mg/ml or 250 mg/ml). The pH of samples of the concentrated formulations was measured, the samples were incubated at 40° C., and the percentage (%) of HMW species was measured at several different times following incubation. The increase in aggregation as reflected by the percentage of HMW species after a one-month incubation is shown in FIG. 3A. The available data showed a minimum in increase of HMW species at a pH value of approximately 5.7. In the presence of sucrose, protein aggregation is minimized at a pH value of approximately pH 5.7 (FIG. 3B).

### Example 5

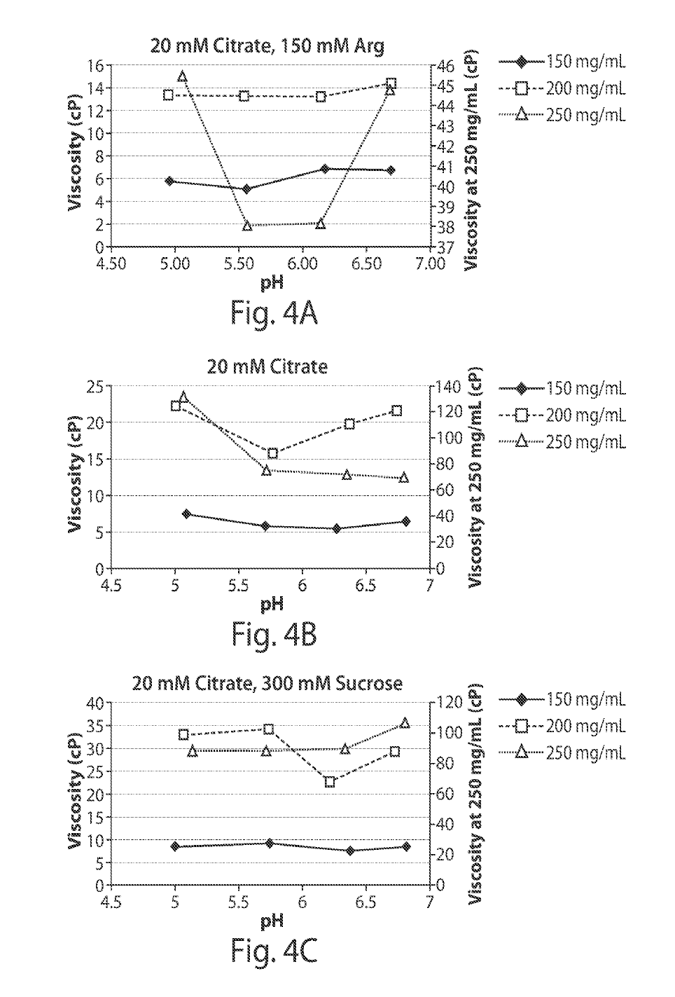

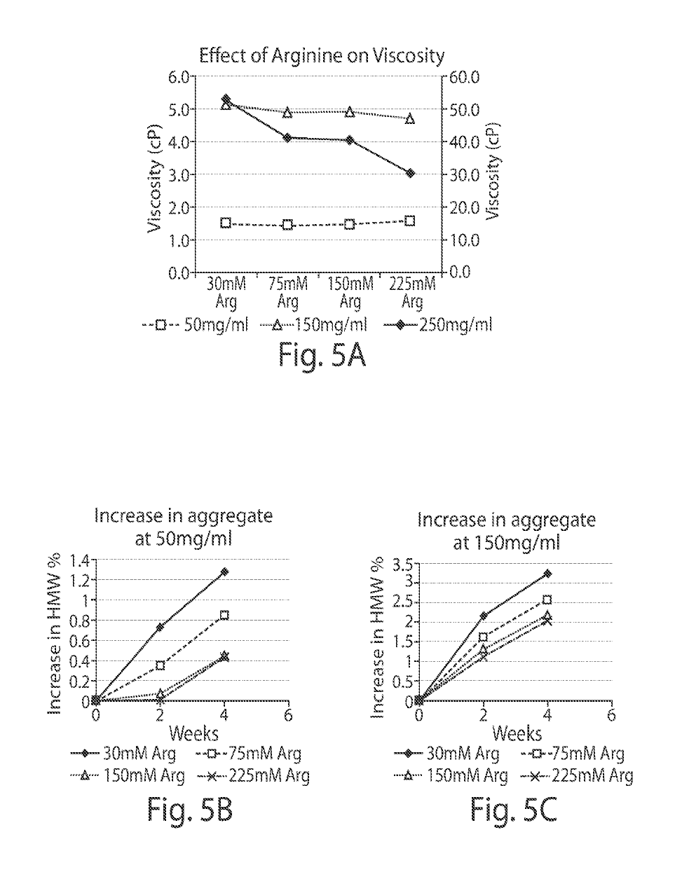

Anti-CD40 antibody was dialyzed into 20 mM citrate buffer at pH values of 5.0, 5.7, 6.4 or 7.0, with (1) no additional excipient, (2) 150 mM arginine (Arg) or (3) 300 mM sucrose. Following dialysis, each formulation was concentrated (e.g., to 150 mg/ml, 200 mg/ml or 250 mg/ml). The viscosity was measured using an mVROC viscometer. In each case, the viscosity increased with increasing protein concentration. In the presence of arginine (FIG. 4A), at the highest antibody concentration tested (250 mg/ml), the viscosity of the formulation was minimized at a pH value of approximately 5.7. At lower concentrations (e.g., 150 mg/ml and 200 mg/ml) no clear trend was observed, although viscosities trended lower between pH values of 5.0 and 6.0. In the presence of citrate buffer only (FIG. 4B), the formulation having an anti-CD40 antibody concentration of 200 mg/ml showed most clearly a viscosity minimum at a pH value of approximately 5.7, with a similar trend at an anti-CD40 antibody concentration of 150 mg/ml. In the formulation having an anti-CD40 antibody concentration of 250 mg/ml, viscosity was lowest between pH values of 5.7 and 6.8. For formulations containing 300 mM sucrose (FIG. 4C), the data suggest that the viscosity is minimized between pH 5.5-6.3.

### Example 6

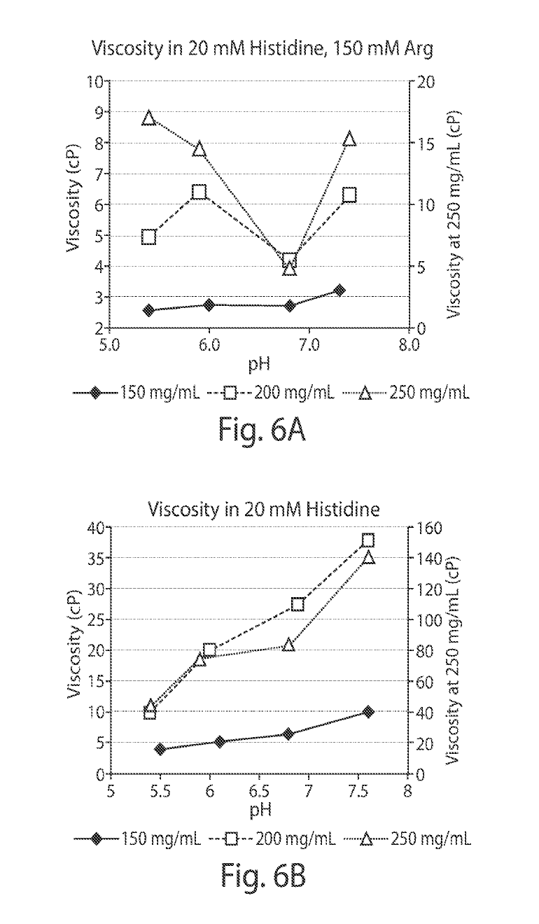

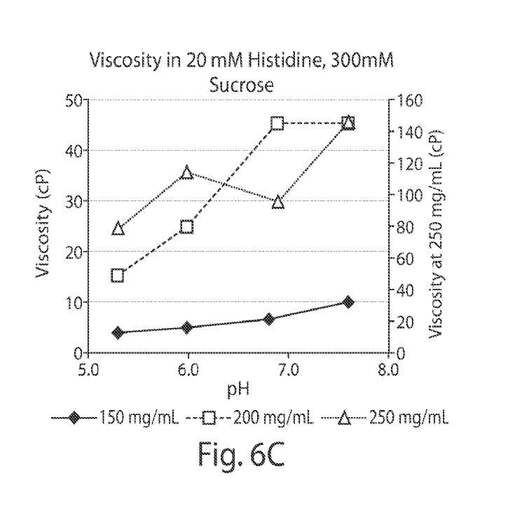

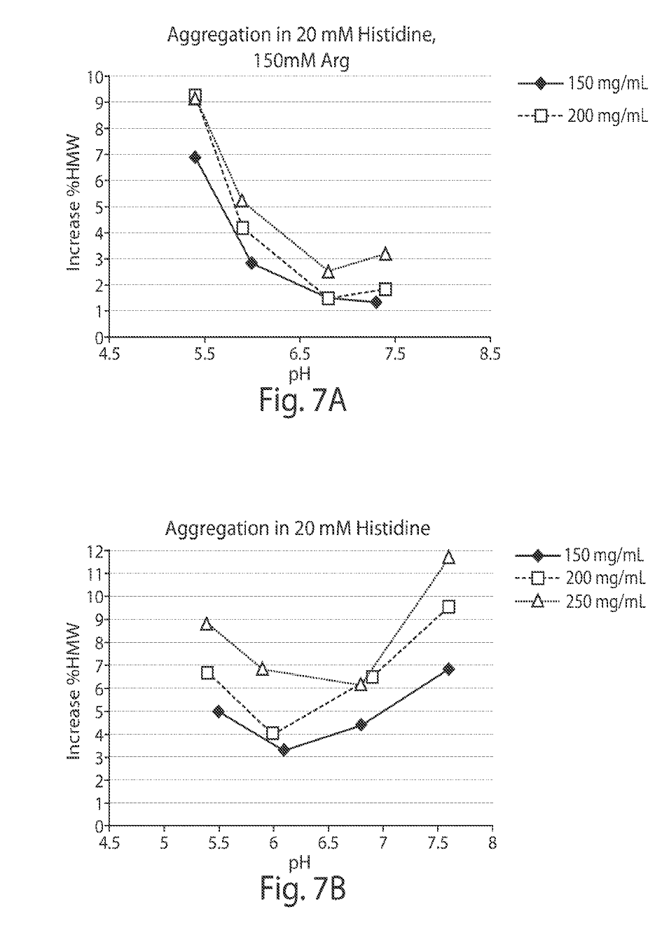

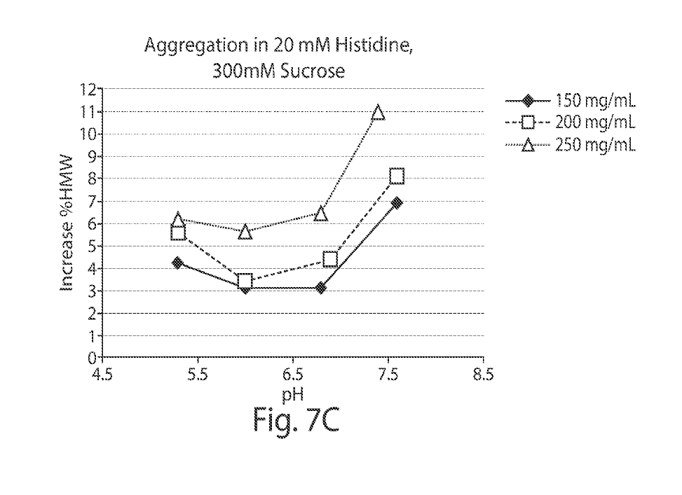

Anti-CD40 antibody was dialyzed into 20 mM histidine buffer at pH values of 5.0, 5.7, 6.4 or 7.0, with (1) no additional excipient, (2) 150 mM arginine (Arg) or (3) 300 mM sucrose. Following dialysis, each formulation was concentrated (e.g., to 150 mg/ml, 200 mg/ml or 250 mg/ml). The viscosity was measured using an mVROC viscometer. In each case, the viscosity increased with increasing protein concentration. In the presence of arginine (FIG. 6A), at the highest antibody concentration tested (250 mg/ml), the viscosity of the formulation was minimized at a pH value of approximately 6.7. At the 200 mg/ml antibody concentration, the viscosity was minimized at a pH value of approximately 6.7. At the lowest antibody concentration 150 mg/ml, no clear trend was observed, although viscosities trended lower between pH values of 5.5 and 7.0. In the presence of histidine buffer only (FIG. 6B), the formulations having an anti-CD40 antibody concentration of 250 mg/ml or 200 mg/ml showed most clearly a viscosity minimum at a pH value of approximately 5.4. In the formulation having an anti-CD40 antibody concentration of 150 mg/ml, viscosity was lowest at a pH value of 5.5. For formulations containing 300 mM sucrose (FIG. 6C), the data suggest that the viscosity is minimized between pH 5.0 and 7.0.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An anti-CD40 antibody formulation, comprising:
anti-CD40 antibody;
10 mM to 30 mM citrate; and
140 mM to 160 mM arginine, wherein the formulation in solution has a pH value of 5.0 to 6.5.

2. The anti-CD40 antibody formulation of claim 1, wherein the formulation has an anti-CD40 antibody concentration of 100 mg/ml to 300 mg/ml.

3. The anti-CD40 antibody formulation of claim 1, wherein the formulation has an anti-CD40 antibody concentration of at least 150 mg/ml.

4. The anti-CD40 antibody formulation of claim 3, wherein the formulation has an anti-CD40 antibody concentration of at least 200 mg/ml.

5. The anti-CD40 antibody formulation of claim 4, wherein the formulation has an anti-CD40 antibody concentration of at least 250 mg/ml.

6. The anti-CD40 antibody formulation of any one of claims 1-5, wherein the formulation has an anti-CD40 antibody concentration of 250 mg/ml.

7. The anti-CD40 antibody formulation of any one of claims 1-6, wherein the anti-CD40 antibody is a monoclonal antibody.

8. The anti-CD40 antibody formulation of any one of claims 1-7, wherein the formulation has a citrate concentration of 20 mM.

9. The anti-CD40 antibody formulation of any one of claims 1-8, wherein the formulation has an arginine concentration of 150 mM.

10. The anti-CD40 antibody formulation of any one of claims 1-9, wherein the formulation has a pH value of 5.5-6.0.

11. The anti-CD40 antibody formulation of claim 10, wherein the formulation has a pH value of 5.7.

12. The anti-CD40 antibody formulation of any one of claims 1-11, further comprising sucrose.

13. The anti-CD40 antibody formulation of claim 12, wherein the formulation has a sucrose concentration of 200 mM to 400 mM.

14. The anti-CD40 antibody formulation of claim 13, wherein the formulation has a sucrose concentration of 300 mM.

15. The anti-CD40 antibody formulation of any one of claims 1-14, wherein the increase in the percentage of high molecular weight species present in the formulation after storage for one month at 40° C. is less than 4.

16. The anti-CD40 antibody formulation of any one of claims 1-15, wherein the formulation has a viscosity suitable for subcutaneous injection.

17. The anti-CD40 antibody formulation of any one of claims 1-16, wherein the formulation has a viscosity suitable for injection through a needle having a size of 29 gauge to 31 gauge.

18. The anti-CD40 antibody formulation of any one of claims 1-17, wherein the formulation has a viscosity suitable for injection through a needle having a size of 29 gauge to 31 gauge while at a temperature in range of 25° C. to 40° C.

19. The anti-CD40 antibody formulation of any one of claims 1-18, wherein the formulation has a viscosity of less than 40 cP at anti-CD40 antibody concentrations of up to 250 mg/ml.

20. An anti-CD40 antibody formulation, comprising:
100 mg/ml to 250 mg/ml anti-CD40 antibody; and
buffer having a pH value of 5.5 to 6.0.

21. The anti-CD40 antibody formulation of claim 20, wherein the buffer is a citrate buffer.

22. The anti-CD40 antibody formulation of claim 21, wherein the citrate buffer has a concentration of 10 mM to 30 mM.

23. The anti-CD40 antibody formulation of claim 22, wherein the citrate buffer has a concentration of 20 mM.

24. The anti-CD40 antibody formulation of any one of claims 20-23, further comprising arginine.

25. The anti-CD40 antibody formulation of claim 24, wherein the arginine is present at a concentration of 140 mM to 160 mM.

26. The anti-CD40 antibody formulation of claim 25, wherein the arginine is present at a concentration of 150 mM.

27. The anti-CD40 antibody formulation of any one of claims 20-26, further comprising sucrose.

28. The anti-CD40 antibody formulation of claim 27, wherein the sucrose is present at a concentration of 200 mM to 400 mM.

29. The anti-CD40 antibody formulation of claim 28, wherein the sucrose is present at a concentration of 300 mM.

30. An anti-CD40 antibody formulation, comprising:
at least 150 mg/ml anti-CD40 antibody;
20 mM buffer containing sodium citrate and citric acid; and
150 mM arginine, wherein the formulation has a pH value of 5.5 to 6.0.

31. The anti-CD40 antibody formulation of claim 30, further comprising 300 mM sucrose.

32. A method of producing an anti-CD40 antibody formulation, the method comprising:
combining 100 mg/ml to 300 mg/ml anti-CD40 antibody in citrate buffer; and
adjusting pH of the formulation to 5.0 to 6.5.

33. The method of claim 32, wherein the citrate buffer has a concentration of 10 mM to 30 mM.

34. The method of claim 33, wherein the citrate buffer has a concentration of 20 mM.

35. An anti-CD40 antibody formulation, comprising:
anti-CD40 antibody;
10 mM to 30 mM histidine; and
140 mM to 160 mM arginine, wherein the formulation in solution has a pH value of 5.0-6.5.

36. The anti-CD40 antibody formulation of claim 35, wherein the formulation has an anti-CD40 antibody concentration of 100 mg/ml to 300 mg/ml.

37. The anti-CD40 antibody formulation of claim 35, wherein the formulation has an anti-CD40 antibody concentration of at least 150 mg/ml.

38. The anti-CD40 antibody formulation of claim 37, wherein the formulation has an anti-CD40 antibody concentration of at least 200 mg/ml.

39. The anti-CD40 antibody formulation of claim 38, wherein the formulation has an anti-CD40 antibody concentration of at least 250 mg/ml.

40. The anti-CD40 antibody formulation of any one of claims 35-39, wherein the formulation has an anti-CD40 antibody concentration of 250 mg/ml.

41. The anti-CD40 antibody formulation of any one of claims 35-40, wherein the anti-CD40 antibody is a monoclonal antibody.

42. The anti-CD40 antibody formulation of any one of claims 35-41, wherein the formulation has a histidine concentration of 20 mM.

43. The anti-CD40 antibody formulation of any one of claims 35-42, wherein the formulation has an arginine concentration of 150 mM.

44. The anti-CD40 antibody formulation of any one of claims 35-43, wherein the formulation has a pH value of 5.5-6.0.

45. The anti-CD40 antibody formulation of claim 44, wherein the formulation has a pH value of 5.7.

46. The anti-CD40 antibody formulation of any one of claims 35-45, further comprising sucrose.

47. The anti-CD40 antibody formulation of claim 46, wherein the formulation has a sucrose concentration of 200 mM to 400 mM.

48. The anti-CD40 antibody formulation of claim 47, wherein the formulation has a sucrose concentration of 300 mM.

49. The anti-CD40 antibody formulation of any one of claims 35-48, wherein the increase in the percentage of high molecular weight species present in the formulation after storage for one month at 40° C. is less than 4.

50. The anti-CD40 antibody formulation of any one of claims 35-49, wherein the formulation has a viscosity suitable for subcutaneous injection.

51. The anti-CD40 antibody formulation of any one of claims 35-50, wherein the formulation has a viscosity suitable for injection through a needle having a size of 29 gauge to 31 gauge.

52. The anti-CD40 antibody formulation of any one of claims 35-51, wherein the formulation has a viscosity suitable for injection through a needle having a size of 29 gauge to 31 gauge while at a temperature in range of 25° C. to 40° C.

53. The anti-CD40 antibody formulation of any one of claims 35-52, wherein the formulation has a viscosity of less than 40 cP at anti-CD40 antibody concentrations of up to 250 mg/ml.

54. An anti-CD40 antibody formulation, comprising:
100 mg/ml to 250 mg/ml anti-CD40 antibody; and
buffer having a pH value of 5.5 to 6.0.

55. The anti-CD40 antibody formulation of claim 54, wherein the buffer is a histidine buffer.

56. The anti-CD40 antibody formulation of claim 55, wherein the histidine buffer has a concentration of 10 mM to 30 mM.

57. The anti-CD40 antibody formulation of claim 56, wherein the histidine buffer has a concentration of 20 mM.

58. The anti-CD40 antibody formulation of any one of claims 54-57, further comprising arginine.

59. The anti-CD40 antibody formulation of claim 58, wherein the arginine is present at a concentration of 140 mM to 160 mM.

60. The anti-CD40 antibody formulation of claim 59, wherein the arginine is present at a concentration of 150 mM.

61. The anti-CD40 antibody formulation of any one of claims 54-60, further comprising sucrose.

62. The anti-CD40 antibody formulation of claim 61, wherein the sucrose is present at a concentration of 200 mM to 400 mM.

63. The anti-CD40 antibody formulation of claim 62, wherein the sucrose is present at a concentration of 300 mM.

64. An anti-CD40 antibody formulation, comprising:
at least 150 mg/ml anti-CD40 antibody;
20 mM buffer containing histidine; and
150 mM arginine, wherein the formulation has a pH value of 5.5 to 6.0.

65. The anti-CD40 antibody formulation of claim 64, further comprising 300 mM sucrose.

66. A method of producing an anti-CD40 antibody formulation, the method comprising:
combining 100 mg/ml to 300 mg/ml anti-CD40 antibody in histidine buffer; and
adjusting pH of the formulation to 5.5 to 6.5.

67. The method of claim 66, wherein the histidine buffer has a concentration of 10 mM to 30 mM.

68. The method of claim 67, wherein the histidine buffer has a concentration of 20 mM.
